(19) United States
(12) Patent Application Publication
(10) Pub. No.: US 2018/0311175 A1
(43) Pub. Date: Nov. 1, 2018

(54) MULTIFUNCTIONAL BIODEGRADABLE CARRIERS FOR DRUG DELIVERY

(71) Applicant: University of Maryland, College Park, College Park, MD (US)

(72) Inventors: Alexander K. ANDRIANOV, Gaithersburg, MD (US); Alexander MARIN, Rockville, MD (US); Thomas R. FUERST, Darnestown, MD (US); Andre MARTINEZ, Chevy Chase, MD (US)

(21) Appl. No.: 15/772,159

(22) PCT Filed: Oct. 28, 2016
(86) PCT No.: PCT/US16/59516

§ 371 Date: Apr. 30, 2018

Related U.S. Application Data
(60) Provisional application No. 62/247,373, filed on Oct. 28, 2015.

(51) Int. Cl. A61K 9/51; A61K 47/58

(52) U.S. Cl. CPC A61K 9/5146; C08G 79/025; A61K 47/605

(57) ABSTRACT

Provided are pharmaceutical agent carriers (e.g., multifunctional polyphosphazenes). Such polymers can be useful as delivery carriers for pharmaceutical agents. Specifically they can be useful for prolonging serum half-life, reducing immunogenicity, and facilitating intracellular and cytosolic delivery of pharmaceutical agents. Also provided are compositions comprising pharmaceutical agent carriers and methods of delivering pharmaceutical agents using the compositions.

### CROSS REFERENCE TO RELATED APPLICATIONS

This application claims priority to U.S. Provisional Application No. 62/247,373, filed on Oct. 28, 2015, the disclosure of which is hereby incorporated by reference.

### FIELD OF THE DISCLOSURE

The disclosure generally relates to delivery of pharmaceutical agents using biodegradable carriers. More particularly the disclosure generally relates to biodegradable carriers having multifunctional macromolecular domains.

### BACKGROUND OF THE DISCLOSURE

The demand for novel multifunctional systems for the delivery of pharmaceutical agents stems out of the pressing need to improve the efficacy and reduce toxicity of drugs. Some of the key objectives in the development of delivery carriers include prolongation of serum half-life, reduction of immunogenicity, and facilitation of intracellular and cytosolic delivery of pharmaceutical agents.

The majority of existing delivery technologies focus either on stabilization of pharmaceutical agents by making them invisible to the immune system and protecting them against opsonization, or on targeting drugs to specific tissues, cells or subcellular compartments, such as cytosol. For example, PEGylation technology is designed to form steric ‘nano-shell’ around the protein protecting it from being recognized by a body's immune system. It relies on covalent modification of a non-biodegradable water-soluble polymer-polyethylene glycol (PEG). This method in its present form, although proven successful for stabilization of a number of protein therapeutics, also suffers from severe limitations. The approach, which relies on a covalent attachment of PEG to a protein, requires sophisticated synthetic routes. This can lead to a reduction of avidity, and may also result in toxic or undesirable residuals. Production of such protein-PEG conjugates require sophisticated technologies and equipment, multiple step processes and dictate high development and manufacturing costs. In its present form it also does not allow for facilitation of cellular internalization of pharmaceutical agent, their cytosolic delivery, and is scarcely compatible with targeting mechanisms.

Thus, there is a clear need for novel multifunctional pharmaceutical drug delivery technologies allowing a simple formulation approach and capable of integrating stabilization and cellular delivery modalities.

### SUMMARY OF THE DISCLOSURE

In an aspect, the present disclosure provides multifunctional macromolecular carriers. The multifunctional carriers can comprise one or more pharmaceutical agents.

In accordance with an aspect of the present disclosure there is provided a multifunctional macromolecular carrier for the delivery of one or more pharmaceutical agents comprising a hydrophilic macromolecular domain of essentially linear geometry and a biodegradable macromolecular domain, comprising at least one side group selected from the following functionalities:

(1) ligands providing binding affinity to a pharmaceutical agent,
(2) functionalities displaying membrane disruptive activity between pH 4.0 and pH 6.8,
wherein said hydrophilic macromolecular domain can be linked to said biodegradable macromolecular domain through covalent bonds or non-covalent interactions.

In the preferred embodiment said hydrophilic macromolecular domain is poly(ethylene glycol) and said biodegradable macromolecular domain is polyphosphazene. In the most preferred embodiment said domains are linked through one or more covalent bonds.

In an aspect, the present disclosure provides comprising one or more multifunctional macromolecular carriers of the present disclosure that can, optionally, comprise one or more pharmaceutical agents. For example, a composition also comprises a pharmaceutically acceptable carrier.

In an aspect, the present disclosure provides uses of multifunctional macromolecular carriers of the present disclosure. For example, the carriers can be used to delivery one or more pharmaceutical agents to an individual.

For example, a method of delivering a pharmaceutical agent to an individual in need of a pharmaceutical agent comprising administering one or more multifunctional macromolecular carriers comprising one or more pharmaceutical agents of the present disclosure or one or more compositions of the present disclosure to an individual in need of the pharmaceutical agent.

### BRIEF DESCRIPTION OF THE FIGURES

For a fuller understanding of the nature and objects of the disclosure, reference should be made to the following detailed description taken in conjunction with the accompanying figures.

FIG. 1 shows a schematic presentation of multifunctional biodegradable carrier.

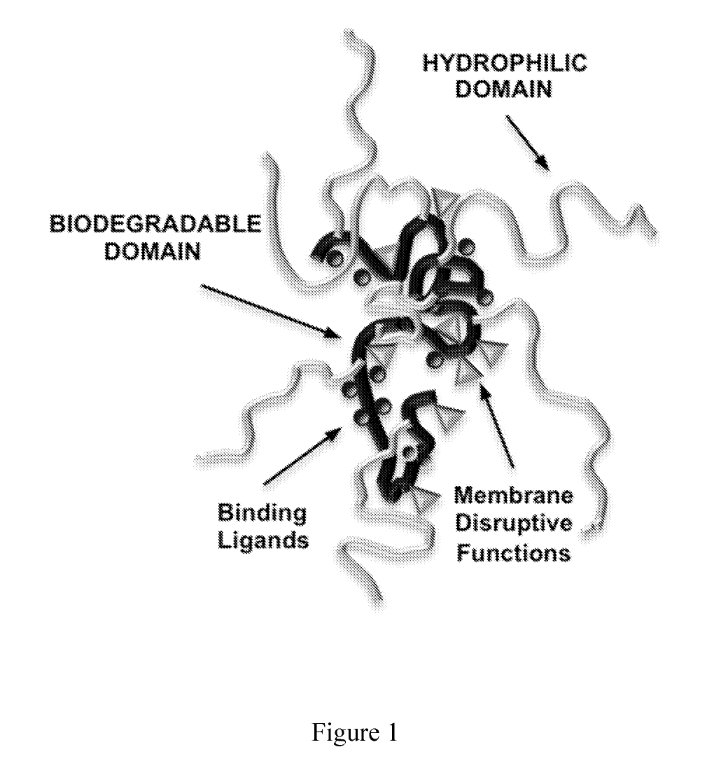

FIG. 2 shows hydrodynamic diameters (as determined by dynamic light scattering) (circles) and zeta potentials (triangles) of the non-covalently bound PCPP-PEG carriers as a function of PEG concentration (0.025 mg/mL PCPP, PBS, pH 7.1).

FIG. 3 shows loading (circles) and efficiency (triangles) of Cytochrome C binding by non-covalently assembled PCPP-PEG carrier (closed symbols) as a function of protein concentration (0.025 mg/mL PCPP, 0.1 mg/ml PEG, PBS, pH 7.4). Binding parameters of PCPP (open symbols) at the same conditions are shown for comparison.

FIG. 4 shows membrane disruptive properties of non-covalently bound PCPP-PEG complexes as a function of pH (0.025 mg/mL PCPP, PBS, molecular weight of PEG 100,000 g/mol).

FIG. 5 shows membrane disruptive properties of non-covalently bound PCPP-PEG complexes as a function of PEG concentration (0.025 mg/mL PCPP, PBS, pH 6.5, molecular weight of PEG 100,000 g/mol).

FIG. 6 shows membrane disruptive properties of PCEP domain as a function of pH (0.025 mg/mL PCEP, 0.025 mg/mL PCPP, PBS).

FIG. 7 shows membrane disruptive properties of PCAP-20, PCAP-40, and PCAP-70 as a function of pH (0.05 mg/mL, 50 mM PBS for pH>5, 50 mM citric acid/Na₂HPO₄ for pH<5.0).

FIG. 8 shows hydrolytic degradation of PCAP-20, PCAP-40, and PCAP-70 (0.5 mg/mL, PBS). Squares: PCAP-70, diamonds: PCAP-40, crosses: PCAP-20

FIG. 9 shows avidin binding by copolymers PCAP-20, PCAP-40, and PCAP-70 as measured by AF4. The results are expressed as the number of protein molecules per polymer chain (0.015 mg/mL polymer, 0.1 mg/mL avidin, PBS).

FIG. 10 shows self-assembly of PCAP-20, PCAP-40, and PCAP-70 (A: 0.1 mg/mL, PBS; B: 0.1 mg/mL polymer, 4.5 mg/mL spermidine trihydrochloride).

### DETAILED DESCRIPTION OF THE DISCLOSURE

Although claimed subject matter will be described in terms of certain embodiments and examples, other embodiments and examples, including embodiments and examples that do not provide all of the benefits and features set forth herein, are also within the scope of this disclosure. Various structural, logical, process step, and electronic changes may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

The present disclosure provides multifunctional macromolecular carriers. The multifunctional carriers can comprise one or more pharmaceutical agents. The multifunctional macromolecular carriers can be used in methods of delivering pharmaceutical agents to individuals.

The multifunctional macromolecular carriers of the present disclosure are an alternative to previous PEGylation techniques and avoids undesirable chemical conjugations of drugs with poly(ethylene glycol) (PEG). The multifunctional macromolecular carriers can attach to a pharmaceutical agent non-covalently through spontaneous self-assembly in aqueous solution and afford protective properties to the drug. This can potentially result in one or more of the following: (i) innovative “mix-and-use” formulation approach to stabilization of macromolecular drug, (ii) broad scope of pharmaceutical agents, to which the technology can be applied, (iii) contaminant free formulations, (iv) prolonged half-life, and (v) dramatic manufacturing labor, equipment, and cost reduction.

In an aspect, the present disclosure provides multifunctional macromolecular carriers. The multifunctional carriers can comprise one or more pharmaceutical agents.

For example, a multifunctional macromolecular carrier comprises (or consists essentially of or consists of): i) a hydrophilic macromolecular domain, and ii) a biodegradable macromolecular domain (e.g., a biodegradable polyphosphazene macromolecular domain). The macromolecular domain can have one or more ligands having binding affinity to a pharmaceutical agent and, optionally, one or more groups having functionalities displaying membrane disruptive activity between pH 4.0 and pH 6.8 and/or one more other side groups. In various examples, the hydrophilic macromolecular domain and the biodegradable polyphosphazene macromolecular domain are linked through one or more covalent bonds or one or more non-covalent interactions. In various examples, the multifunctional carriers further comprise (or consist essentially of or consist of) one or more pharmaceutical agents. The pharmaceutical agents can be bound to the multifunctional macromolecular carrier through one or more multivalent covalent interactions or one or more multivalent non-covalent interactions.

In an example, the hydrophilic molecular domain and/or the biodegradable molecular domain are discrete compounds. In another example, the hydrophilic molecular domain is formed by pendant groups on the biodegradable molecular domain (e.g., the hydrophilic molecular domain is formed by pendant groups on a biodegradable polymer). In another example, a hydrophilic molecular domain is formed by a protonated form a compound or formed by a group or groups formed from a deprotonated form of a compound.

Hydrophilic macromolecular domain that can have essentially linear geometry can be any water-soluble polymer that can be attached either covalently or non-covalently to a biodegradable macromolecular domain. Examples include, but are not limited to, polyvinylpyrrolidone, poly(hydroxypropylmethacrylate), poly(ethylene glycol)-co-poly(propylene glycol), poly(vinyl alcohol), poly(dimethoxyethoxyethoxyphosphazene), and poly[di[2-(2-oxo-1-pyrrolidinyl)ethoxy]phosphazene].

In the preferred embodiment hydrophilic macromolecular chain of essentially linear geometry is a polyether, such as, for example, poly(ethylene glycol). In the most preferred embodiment the macromolecule is poly(ethylene glycol) with the molecular weight of at least 5,000 g/mol. In yet another embodiment, the molecular weight of poly(ethylene glycol) is between 25,000 and 35,000 g/mol. The poly(ethylene glycol) chain can be connected to the biodegradable domain covalently through nitrogen or oxygen atoms or non-covalently, such as, for example, through hydrogen bonds or formation of pseudorotaxanes.

Any biodegradable macromolecule that can be functionalized with either ligands providing binding affinity to a pharmaceutical agent, or functionalities displaying membrane disruptive activity, or combination thereof can serve as a biodegradable macromolecular domain of the present disclosure. Examples, include but are not limited to, are polyphosphates, polyurethanes, polyesters, and polyanhydrides. In the preferred embodiment a biodegradable macromolecular domain of the present disclosure is polyphosphazene.

Polyphosphazenes are polymers with backbones having alternating phosphorus and nitrogen, separated by alternating single and double bonds. Each phosphorous atom is covalently bonded to two pendant groups (“R”). The repeat unit in polyphosphazenes has the following general formula:

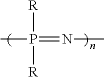

wherein n is an integer. Each R may be the same or different. The pendant groups are also referred to herein as R and R′.

In a non-limiting embodiment, the polyphosphazene has more than three types of pendant groups and the groups vary randomly or regularly throughout the polymer. The phosphorus thus can be bound to two like groups, or to two different groups.

In an embodiment the polyphosphazene is not linked to N,N-diisopropylethylenediamine (DPA). In embodiments compositions of the disclosure are DPA free.

In a non-limiting embodiment, the polymers of the present disclosure may be prepared by producing initially a reactive macromolecular precursor such as, but not limited to, poly(dichlorophosphazene). The pendant groups then are substituted onto the polymer backbone by reaction between the reactive chlorine atoms on the backbone and the appropriate organic nucleophiles, such as, for example, alcohols, amines, or thiols. Polyphosphazenes with two or more types of pendant groups can be produced by reacting a reactive macromolecular precursor such as, for example, poly(dichlorophosphazene) with two or more types of nucleophiles in a desired ratio. Nucleophiles can be added to the reaction mixture simultaneously or in sequential order. The resulting ratio of pendant groups in the polyphosphazene will be determined by a number of factors, including the ratio of starting materials used to produce the polymer, the order of addition, the temperature at which the nucleophilic substitution reaction is carried out, and the solvent system used. While it is difficult to determine the exact substitution pattern of the groups in the resulting polymer, the ratio of groups in the polymer can be determined easily by one skilled in the art.

In yet another non-limiting embodiment, the multifunctional macromolecular carrier of the present disclosure may be prepared through spontaneous self-assembly of biodegradable polyphosphazene domain and hydrophilic domain using non-covalent interactions, such as, for example, hydrogen bonding, ionic or hydrophobic interactions. The biodegradable polyphosphazene capable of such interactions is contacted with the hydrophilic polymer by simple mixing in aqueous solutions or organic solvents. Aqueous buffer solutions with pH values and ionic strength that enhance such interactions can be employed for desirable results. In the preferred embodiment said non-covalent interactions are multivalent interactions. In the most preferred embodiment polymers produce the pharmaceutical carrier of the present disclosure through hydrogen bonds. The multifunctional macromolecular carrier can then be used as a solution or it can be recovered from the reaction mixture by precipitating, freeze-drying or other methods.

The binding ligands of the present disclosure include functionalities capable of forming covalent or non-covalent links with a therapeutic drug.

In an embodiment the binding of polymeric carrier to a therapeutic drug is through non-covalent interactions, such as, for example, electrostatic, hydrogen bonds, van der Waals forces, and hydrophobic effects. In such case the carrier forms a complex with a drug typically through a spontaneous self-assembly with drug in aqueous solutions.

In a preferred environment, such therapeutic drug—polymer carrier binding is enabled through the establishment of multivalent interactions, such as, for example, ionic, hydrogen bond, receptor—ligand, host-guest inclusion, and peptide—protein interactions. Multivalent interactions are preferred way to achieve effective binding, especially when individual binding interactions are weak. Multivalent interactions are also preferred when ‘flexible’ binding is important between the carrier and the protein drug allowing for the polymer ligand to jump from one binding site to another across a protein surface through a combination of mechanisms that can be likened to “hopping, walking and flying.”

Examples of suitable ligands for multivalent interactions may include ionized carboxyl and tertiary amino groups, hydroxyl, carbonyl, non-ionized carboxyl groups, components of β-cyclodextrin-adamantane pair, pseudorotaxane pairs, such as, for example, α-cyclodextrin-poly(ethylene glycol), α-cyclodextrin-N-alkylpyridinium, and various complexes of cucurbit[n]urils with positively charged hydrophobic guests. Additional examples of ligands include, but are not limited to, short disordered peptides or peptide fragments, which partially mimic the interface area (pockets) of protein drugs. This can be represented by the binding of tyrosyl-phosphorylated peptides to proteins containing Src homology domain 2 (SH2) or phosphotyrosyl binding domain (PTB) domain, binding of peptides with certain proline motifs to proteins containing Src homology domain 3 (SH3).

In yet another embodiment, binding ligands can contain hydrophobic alkyl groups to provide for interactions with poorly soluble drugs.

In an alternative embodiment, the ligands can include functional groups usable for covalent attachment of drug, such as:
amino groups for conjugation reactions using N-hydroxysuccinimide (NHS) esters, imidoester, hydroxymethyl phosphine, guanidination, fluorophenyl esters, carbodiimides, anhydrides, arylating agents, carbonates, aldehydes, and glyoxals;
carboxyl groups for conjugation reactions using carbodiimides,
thiol groups for reactions with maleimide, haloacetyl, pyridyldisulfide, vinyl sulfone;
hydroxyl groups for conjugation reactions using isocyanates, carbonyldiimidazole
aldehyde and ketone groups for conjugation reactions using hydrazine derivative, Schiff base formation, and reductive amination.

Side groups providing pH dependent membrane disruptive activity can include pH sensitive fusogenic peptides of natural (N-terminus of hemagglutinin subunit HA-2 of influenza virus) or synthetic (WEAALAEALAEALAEHLAEALAEALEALAA (GALA), WEAKLAKALAKALAKHLAKALAKALKACEA (KALA)) origin, tertiary amino groups, and carboxylic acid groups.

In an embodiment, the membrane disruptive functionalities include dimethylaminopropyl, imidazole, histidine, quinoline and isoquinoline groups, in which the charges are ‘masked’ at neutral pH. In the preferred embodiment the membrane disruptive functionalities include carboxylatophenoxy side groups. In the most preferred embodiment the membrane disruptive functionalities include carboxylatoethylphenoxy side groups.

In an embodiment, ligands providing binding affinity to a therapeutic drug constitute the same side groups as functionalities displaying membrane disruptive activity between pH 4.0 and pH 6.8. In yet another embodiment these side groups are different.

Other side groups can be used in addition to the groups listed above. They may include hydrophilic side groups to provide for improved solubility of polyphosphazene in aqueous solutions, hydrophobic side groups to increase membrane disruptive activity, smaller pendant groups to provide for better conformational flexibility for macromolecular self-assembly.

In an embodiment other side groups include functionalities useful in cellular or tissue targeting. The appropriate ligands can, for example, include ligands targeting common tumour-enriched antigens, such as, for example, folate receptor (FR)50, prostate-specific membrane antigen (PSMA; also known as FOLH1), glucose trans-porter 1 (GLUT1; also known as SLC2A1), somatostatin receptor 2 (SSTR2), cholecystokinin type B receptor (CCKBR), bombesin receptor, sigma non-opioid intracellular receptor 1 (SIGMAR1) and SIGMAR2, cell-adhesion proteins, such as, for example, intercellular adhesion molecule 1 (ICAM1; also known as CD54), CD44, leukocyte function-associated antigen 1 (LFA1; also known as ITGB2) and CD24 or any other ligand-receptor pairs as described elsewhere.

In yet another embodiment other side groups are hydrolysis sensitizers. The choice of side groups for modulating hydrolytic degradation of polyphosphazene or other macromolecule is determined by the desirable rate of degradation and clearance under physiological conditions and shelf-life requirements. The side groups that can be used to increase the rate of hydrolytic degradation of polyphosphazene carrier may include various esters of amino acids, such as, for example, ethyl glycinate, ethyl alaninate, phenyl alaninate, imidazole. In a preferred environment, the side groups capable of increasing hydrolytic degradation of polyphosphazene are hydrophilic groups, such as, for example, oxyethylpyrrolidone or aminopropylpyrrolidone.

In an embodiment the molar content of hydrophilic macromolecular chain of essentially linear geometry does not exceed 40% mol. In the preferred embodiment, the molar content of hydrophilic macromolecular domain is between 5 and 20% mol. In another embodiment, the content of binding ligands is between 5 and 60% mol, preferably between 20 and 30% mol. In yet another embodiment, the content of membrane destabilizing groups is between 10 and 40%, preferably between 25 and 35% mol.

In a non-limiting embodiment, the polyphosphazene polymer has an overall molecular weight of 5,000 g/mol to 10,000,000 g/mol, and in another embodiment from 40,000 g/mol to 1,000,000 g/mol.

Formulations for the treatment of diseases in humans comprising a multifunctional macromolecular carrier for the delivery of pharmaceutical agent comprising a hydrophilic macromolecular domain of essentially linear geometry and a biodegradable macromolecular domain, comprising at least one side group selected from the following functionalities:

(1) ligands providing binding affinity to a pharmaceutical agent,
(2) functionalities displaying membrane disruptive activity between pH 4.0 and pH 6.8,
wherein said hydrophilic macromolecular domain can be linked to said biodegradable macromolecular domain through covalent bonds or non-covalent interactions and said macromolecular carrier is formulated with a pharmaceutical agent.

The multifunctional macromolecular carriers can further comprise one more pharmaceutical agents. In an embodiment, there is no covalent bond between the pharmaceutical agent and hydrophilic molecular domain (e.g., poly(ethylene glycol) or poly(ethylene glycol group)). In an embodiment, a pharmaceutical agent (e.g., a small molecule, nucleic acid, peptide or protein) is not a hydrophobic pharmaceutical agent. In an embodiment, a pharmaceutical agent (e.g., a small molecule, nucleic acid, peptide or protein) is a water-soluble pharmaceutical agent.

In an embodiment, pharmaceutical agents are small molecules. In another embodiment pharmaceutical agent are nucleic acids. In the most preferred embodiment, pharmaceutical agent are protein or peptide drugs. A pharmaceutical agent can be any pharmaceutical agent used for therapy of, for example, cancers, immune disorders, infections, and other diseases.

Examples of protein drugs include, but not limited to antibody-based drugs, Fc fusion proteins, anticoagulants, blood factors, bone morphogenetic proteins, engineered protein scaffolds, enzymes, growth factors, hormones, interferons, interleukins, and thrombolytics.

In the preferred embodiment, the drugs are monoclonal antibodies (MAbs), which include, but not limited to abciximab, rituximab, basiliximab, palivizumab, infliximab, trastuzumab, alemtuzumab, adalimumab, tositumomab-I131, cetuximab, ibrituximab tiuxetan, omalizumab, bevacizumab, natalizumab, ranibizumab, panitumumab, eculizumab, certolizumab pegol, golimumab, canakinumab, catumaxomab, ustekinumab, tocilizumab, ofatumumab, denosumab, belimumab, ipilimumab, brentuximab. In the most preferred embodiment, protein drugs are bispecific Mabs, including, but not limited to bi-specific T-cell engagers (BiTEs) and Dual-Affinity Re-Targeting (DART) mabs.

Examples of nucleic acid drugs include DNA-Based Therapeutics, such as, for example, Oligonucleotides for Antisense and Antigene Applications, Aptamers, DNAzymes and RNA-Based Therapeutics, such as, for example, RNA Aptamers, RNA Decoys, Antisense RNA, Ribozymes, Small Interfering RNAs (siRNAs), and MicroRNA.

Examples of peptide drugs include, but not limited to hormones, neurotransmitters, growth factors, ion channel ligands, and anti-infectives. They include GLP-1 aganists, such as, for example, Byetta™ (exenatide), Bydureon™ (exenatide), Victoza™ (liraglutide), Lyxumia™ (lixisenatide), and most recently Tanzeum™ (albiglutide), Cpd86, ZPGG-72, MOD-6030, ZP2929, HM12525A, VSR859, NN9926, TTP273/TTP054, ZYOG1, MAR709, TT401, HM11260C, PB1023, Dulaglutide, Semaglutide, ITCA. Multifunctional peptides can include a hybrid of two peptides being bound together like modules either directly or via a linker, conjugates with small molecules, oligoribonucleotides, or antibodies.

Example of small drugs include poorly water-soluble drugs. Suitable poorly water soluble pharmaceutical agents include, but are not limited to, taxanes (such as, for example, paclitaxel, docetaxel, ortataxel and other taxanes), epothilones, camptothecins, colchicines, geladanamycins, amiodarones, thyroid hormones, amphotericin, corticosteroids, propofol, melatonin, cyclosporine, rapamycin (sirolimus) and derivatives, tacrolimus, mycophenolic acids, ifosfamide, vinorelbine, vancomycin, gemcitabine, thiotepa, bleomycin, and diagnostic radiocontrast agents.

A multifunctional macromolecular carrier comprising one or more pharmaceutical agents of the present disclosure can be water-soluble. For example, a multifunctional macromolecular carrier comprising one or more pharmaceutical agents of the present disclosure can provide a homogenous aqueous solution.

A multifunctional macromolecular carrier comprising one or more pharmaceutical agents of the present disclosure can be provided in pharmaceutical compositions for administration by combining them with any suitable pharmaceutically acceptable carriers, excipients and/or stabilizers. Examples of pharmaceutically acceptable carriers, excipients and stabilizer can be found in Remington: The Science and Practice of Pharmacy (2005) 21st Edition, Philadelphia, Pa. Lippincott Williams & Wilkins. For example, suitable carriers include excipients, or stabilizers which are nontoxic to recipients at the dosages and concentrations employed, and include buffers such as, for example, acetate, Tris, phosphate, citrate, and other organic acids; antioxidants including ascorbic acid and methionine; preservatives such as, for example, octadecyldimethylbenzyl ammonium chloride; hexamethonium chloride; benzalkonium chloride, benzethonium chloride; phenol, butyl or benzyl alcohol; alkyl parabens such as, for example, methyl or propyl paraben; catechol; resorcinol; cyclohexanol; 3-pentanol; and m-cresol; amino acids such as, for example, glycine, glutamine, asparagine, histidine, arginine, or lysine; monosaccharides, disaccharides, and other carbohydrates including glucose, mannose, or dextrins; chelating agents such as, for example, EDTA; tonicifiers such as, for example, trehalose and sodium chloride; sugars such as, for example, sucrose, mannitol, trehalose or sorbitol; surfactant such as, for example, polysorbate; salt-forming counter-ions such as, for example, sodium; and/or non-ionic surfactants such as, for example, Tween or polyethylene glycol (PEG). The pharmaceutical compositions may comprise other therapeutic agents. The present compositions can be provided as single doses or in multiple doses covering the entire or partial treatment regimen. The compositions can be provided in liquid, solid, semi-solid, gel, aerosolized, vaporized, or any other form from which it can be delivered to an individual.

Formulations can contain other excipients, such as, for example, excipients required to maintain desirable activity or stability of the therapeutic drugs. Excipients can be used also to modulate binding interactions between biodegradable domain of the present disclosure and pharmaceutical agent or enhance biological activity of the formulation. Example of such excipients include non-ionic surfactants, such as, for example, polysorbate (Tween), viscosity enhancers, such as, for example, poly(ethylene glycol) or polyvinylpyrrolidone, various buffers, or stabilizers, such as, for example, trehalose.

In an aspect, the present disclosure provides uses of multifunctional macromolecular carriers of the present disclosure. For example, the carriers can be used to delivery one or more pharmaceutical agents to an individual.

For example, a method of delivering a pharmaceutical agent to an individual in need of a pharmaceutical agent comprising administering one or more multifunctional macromolecular carriers comprising one or more pharmaceutical agents of the present disclosure or one or more compositions of the present disclosure to an individual in need of the pharmaceutical agent.

In various examples, disclosure comprises administering a therapeutically effective amount of a composition described herein. The term “therapeutic” as used herein means a treatment and/or prophylaxis. The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound or composition that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated. Compositions of the disclosure can be administered in conjunction with any conventional treatment regimen, including sequential or simultaneous administration of other agent(s) that are intended to treat or prevent a disease or disorder.

An individual can be a human or non-human animal. Examples of non-human animals include, but are not limited to, dogs, cats, horses, cows, sheep, pigs, chickens, and the like).

Administration of formulations/compositions of the present disclosure as described herein can be carried out using any suitable route of administration known in the art. For example, the compositions/compositions can be administered via intravenous, intramuscular, intraperitoneal, intracerobrospinal, subcutaneous, intra-articular, intrasynovial, oral, topical, or inhalation routes. The compositions may be administered parenterally or enterically. The compositions may be introduced as a single administration or as multiple administrations or may be introduced in a continuous manner over a period of time. For example, the administration(s) can be a pre-specified number of administrations or daily, weekly or monthly administrations, which may be continuous or intermittent, as may be clinically needed and/or therapeutically indicated.

In the following Statements, various examples of multifunctional macromolecular domains of the present disclosure and uses thereof are described:

Statement 1. A multifunctional macromolecular carrier comprising (or consisting essentially of or consisting of): i) a hydrophilic macromolecular domain, and ii) a biodegradable polyphosphazene macromolecular domain comprising one or more ligands having binding affinity to a pharmaceutical agent, where the hydrophilic macromolecular domain and the biodegradable polyphosphazene macromolecular domain are linked through one or more covalent bonds or one or more non-covalent interactions. Optionally, the multifunctional macromolecular carrier also comprises (or also consists essentially of or also consists of) one or more additional macromolecular domains (e.g., hydrophilic domain(s), domain(s) formed from ligands having membrane disrupting activity between pH 4.0 and 6.8, domains formed from other side groups, or a combination thereof).
Statement 2. A pharmaceutical drug carrier according to Statement 1, wherein the biodegradable polyphosphazene macromolecular domain is a polyphosphazene having the following structure:

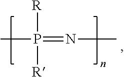

where n is an integer from 10 to 500,000, including all integer number values and ranges therebetween, and at least one R or R′ group is a ligand having binding affinity to a pharmaceutical agent.
Statement 3. A multifunctional macromolecular carrier according to any one of Statements 1 or 2, where the R and R′ groups are at each occurrence in the polyphosphazene selected from ligands having binding affinity to a pharmaceutical agent and ligands having membrane disrupting activity between pH 4.0 and 6.8.
Statement 4. A multifunctional macromolecular carrier according to any one of the preceding Statements, where R and R′ are at each occurrence in the polyphosphazene macromolecular domain are independently selected from:

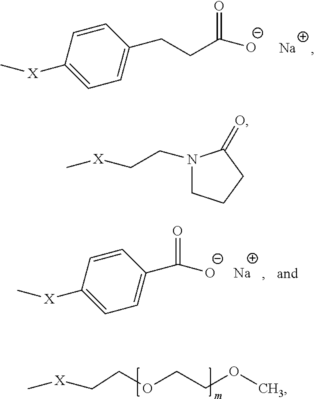

where X is —O— or —NH— and m is between 3 and 1,000, including all integer number values and ranges therebetween.
Statement 5. A multifunctional macromolecular carrier according to any one of the preceding Statements, where the hydrophilic macromolecular domain is selected from poly(ethylene glycol), polyvinylpyrrolidone, poly(hydroxypropylmethacrylate), poly(ethylene glycol)-co-poly(propylene glycol), poly(vinyl alcohol), poly[di(methoxyethoxy)phosphazene], poly[di[2-(2-oxo-1-pyrrolidinyl)ethoxy]phosphazene, poly[di(methoxyethoxyethoxy)phosphazene] and combinations thereof.
Statement 6. A multifunctional macromolecular carrier according to any one of the preceding Statements, where the hydrophilic macromolecular domain is poly(ethylene glycol).
Statement 7. A multifunctional macromolecular carrier according to any one of the preceding Statements, where the hydrophilic macromolecular domain is less than or equal to 40 mole percent of the multifunctional macromolecular carrier and/or the biodegradable domain is greater than or equal to 60 mole percent of the multifunctional macromolecular carrier.
Statement 8. A multifunctional macromolecular carrier according to any one of the preceding Statements, where the hydrophilic macromolecular domain is between 5 and 20 mole percent of the multifunctional macromolecular carrier and/or the biodegradable domain between 95 and 80 mole percent or from 95 to 80 mole percent of the multifunctional macromolecular carrier.
Statement 9. A multifunctional macromolecular carrier according to any one of the preceding Statements, where the multifunctional macromolecular carrier further comprises one or more pharmaceutical agents.
Statement 10. A multifunctional macromolecular carrier according to any one of the preceding Statements, where the pharmaceutical agent is a small molecule drug or combination of small molecule drugs.
Statement 11. A multifunctional macromolecular carrier according to any one of the preceding Statements, where the pharmaceutical agent is selected from nucleic acids, peptide drugs, protein drugs, and combinations thereof.
Statement 12. A multifunctional macromolecular carrier according to any one of the preceding Statements, where the pharmaceutical agent is bound to the multifunctional macromolecular carrier through multivalent non-covalent interactions.
Statement 13. A composition comprising one or more multifunctional macromolecular carriers of any one of Statements 1 to 12.
Statement 14. A composition of according to Statement 13, where the composition comprises a pharmaceutically acceptable carrier.
Statement 15. A composition according to any one of Statements 13 or 14, where the composition further comprises one or more excipients that facilitates interactions between the pharmaceutical agent and the multifunctional macromolecular carrier.
Statement 16. A composition according to any one of Statements 13 to 15, where the excipient comprises spermine, spermidine, or a combination thereof.
Statement 17. A method of delivering a pharmaceutical agent to an individual in need of a pharmaceutical agent comprising administering one or more multifunctional molecular carriers of any one of Statements 1 to 12 or a composition of any one of Statements 13 to 16 to the individual in need of the pharmaceutical agent.

This disclosure is described with respect to the following examples; it is to be understood, however, that the scope of the present disclosure is not intended to be limited thereby.

### Example 1

This example provides a description of preparation of multifunctional carriers using non-covalent interactions.

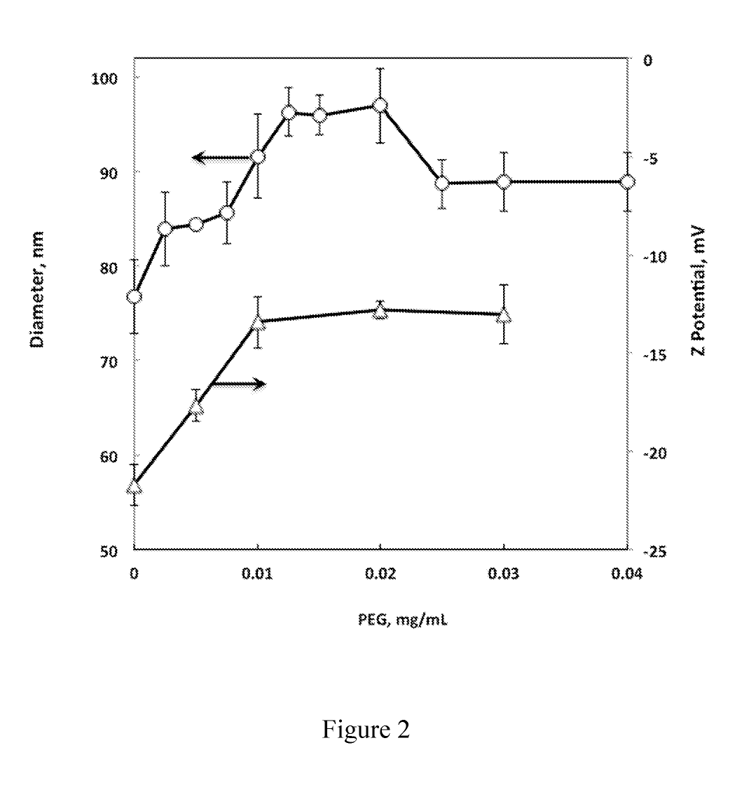

Poly[di(carboxylatophenoxy)phosphazene], PCPP (800,000 g/mol) was used as a biodegradable domain containing benzoic acid side groups as both binding ligands and membrane disruptive functions. PEG (100,000 g/mol) was used as a hydrophilic domain. The PCPP-PEG carrier was prepared through the formation of non-covalent complex between both domains by adding aqueous solutions of PEG to 0.025 mg/mL PCPP solution in aqueous phosphate buffer saline (PBS, pH 7.1). FIG. 2 shows hydrodynamic diameter (as determined by dynamic light scattering) (circles) and zeta potentials (triangles) of the prepared carriers as a function of PEG concentration in solution. The formation of the carrier is manifested through the initial increase in the diameter and decrease in surface charges of the assembly. Some decrease in the size of the carrier in the area of higher PEG concentrations is associated with more compact conformation of the assembly at a larger PEG/PCPP equivalent ratio.

### Example 2

This example provides a description of preparation of multifunctional carrier-protein formulations.

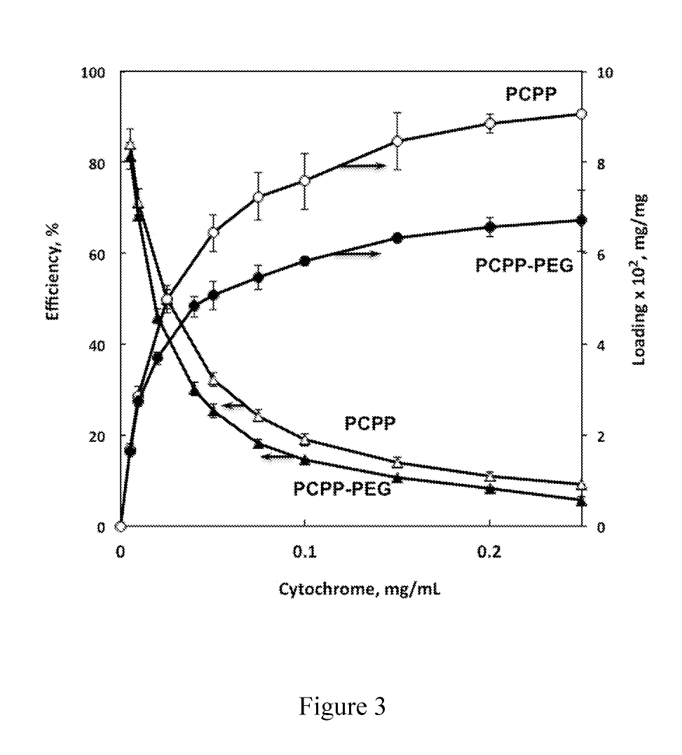

Formulations of PCPP-PEG multifunctional carriers with a model protein drug, Cytochrome C, were prepared as follows. Solutions of Cytochrome C in aqueous PBS (pH 7.4) were added to aqueous solutions of PCPP-PEG carrier, which were prepared as described in Example 1 at PCPP concentration of 0.25 mg/mL and PEG concentration of 0.1 mg/mL (molecular weight 300,000). Concentrations of bound and unbound protein were determined by size exclusion HPLC analysis with UV detection. The loading of protein was calculated as a weight ratio between bound Cytochrome C and a complex and the efficiency of protein binding was defined as a weight ratio between bound and total amount of protein added to the system. FIG. 3 displays these parameters as a function of Cytochrome C concentration in the formulation. As seen from FIG. 3, a multifunctional PCPP-PEG carrier (open symbols) is capable of binding model therapeutic protein. Protein binding ability of PCPP (closed symbols) is shown for comparative purposes.

### Example 3

This example provides a description of pH dependent membrane active properties of non-covalently bound macromolecular carriers.

The membrane disruptive activity of multifunctional carriers, which can be correlated to the ability of the carrier to facilitate endosomal escape and cytosolic delivery of pharmaceutical agent was tested as follows.

100 uL of fresh Porcine Red Blood Cells (RBC) as a 10% suspension in phosphate buffered saline (PBS) (Innovative Technology Inc., Novi, Mich. 48377) was re-suspended in 900 μL of PBS. 50 μL of re-suspended RBC was added to 950 μL of the PCPP-PEG or PCPP formulation in PBS at the appropriate pH, inverted several times for mixing, and incubated in a 37C for 60 min. Cells were then centrifuged at 14,000 rpm for 5 min, and the absorbance of the supernatant was then measured at 541 nm. To determine 100% hemolysis, RBCs were suspended in distilled water and lysed by ultrasound (Branson Sonifier, Model 450). All hemolysis experiments were done in triplicate.

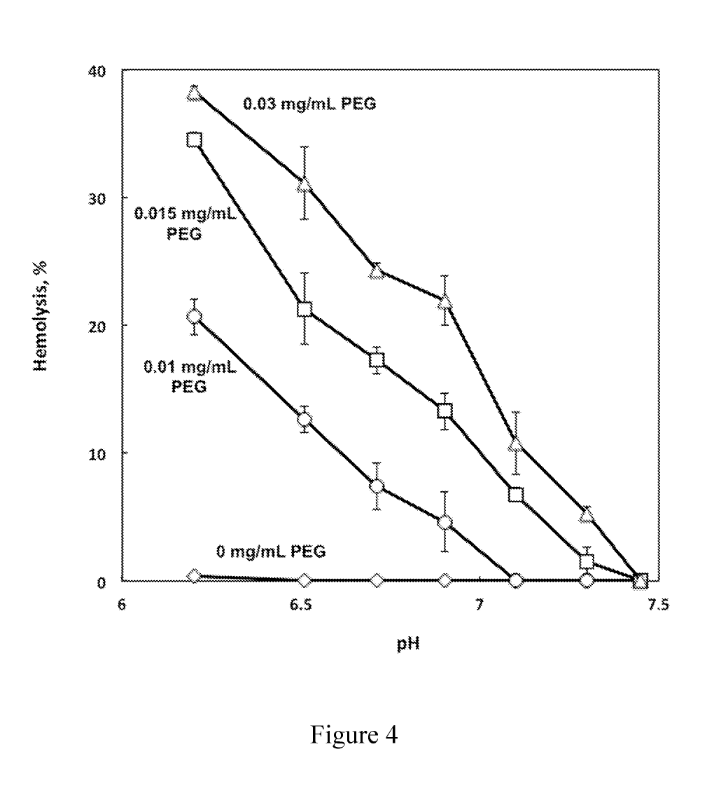

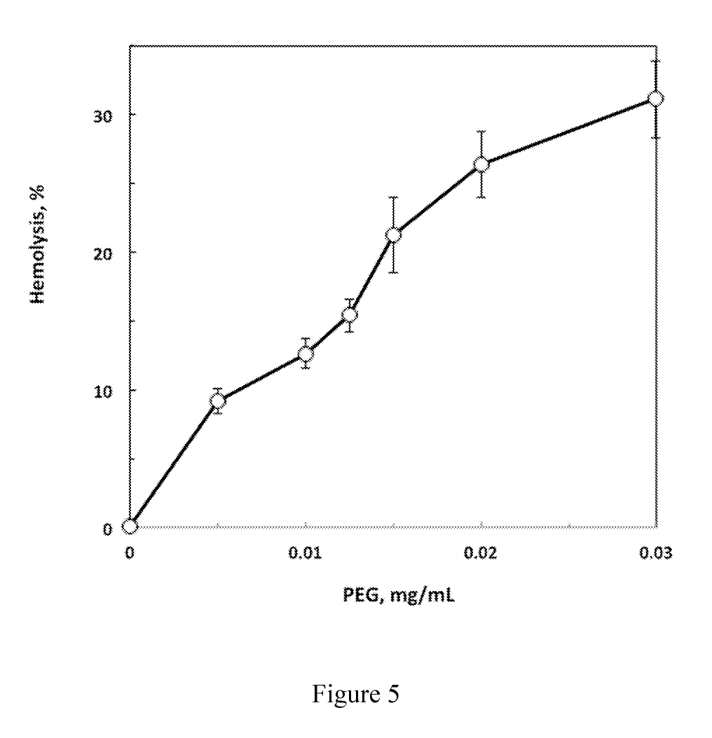

FIG. 4 shows membrane disruptive properties of non-covalently bound PCPP-PEG complex at various PCPP/PEG ratios as a function of pH (0.025 mg/mL PCPP, PBS, molecular weight of PEG 100,000 g/mol). As seen from the figure, PCPP alone did not induce any membrane activity. However, addition of various concentrations of PEG resulted in well-pronounced membrane disruptive properties. The increase in hemolysis correlated both with decrease in pH and increase of PEG content in the complex (FIG. 4 and FIG. 5). As seen from FIG. 4, the pH threshold of activity also increases with the raise in PEG concentration.

### Example 4

This example provides a description of pH dependent membrane active properties of PCEP.

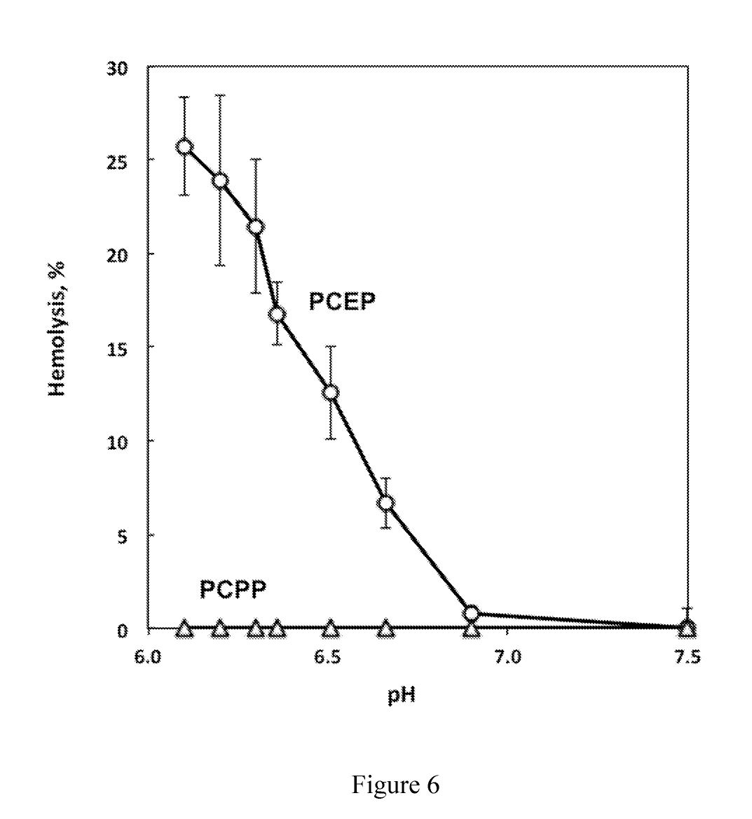

pH dependent membrane disruptive properties of biodegradable polyphosphazene domain containing carboxylatoethylphenoxy side groups (PCEP) were examined as described in Example 3. Concentration of PCEP in PBS was 0.025 mg/mL. The results are shown in FIG. 6. As seen from the figure, PCEP (circles) shows pH dependent membrane activity with a threshold of approximately pH 6.8. The hemolysis rate increases with decrease in pH. PCPP (triangles) does not display membrane activity under the conditions studied and is shown for comparative purposes.

### Example 5

This example provides a description of synthesis of poly[(carboxylatoethylphenoxy)(aminoethylpyrrolidinone)phosphazene], 70/30—PCAP-70.

5 g of Methyl 3(4-hydroxyphenyl)-propionate (MHP) was suspended in deionized water and 0.7 molar equivalents of 6M sodium hydroxide were added. A transparent solution was frozen using dry ice then lyophilized overnight to produce an off white powder of the MHP, sodium salt. 2.0 mL (1.6 mmol) of polydichlorophosphazene (PDCP) solution was added to a three-neck round-bottomed flask under anhydrous conditions and diluted with 8.0 mL of diglyme. 0.3 g (1.7 mmol) of MHP, sodium salt was suspended in 10 mL of diglyme and then added to the flask containing PDCP. The flask was heated to 120° C. with stirring under nitrogen flow, kept at this temperature for 1.5 hours and then allowed to equilibrate to room temperature. 0.465 mL (3.2 mmol) of N-(3′-aminopropyl)-2-pyrrolidinone (APP) was dissolved in 60 mL of 1-methyl-2-pyrrolidinone (NMP) and then added dropwise to the reaction flask while stirring. The reaction mixture was stirred at room temperature overnight. The temperature was then increased to 95° C. and 14 mL of 6M sodium hydroxide was added dropwise with stirring. The reaction mixture was kept on an ice bath to facilitate collection of solid polymer. The supernatant was decanted and the polymer was dissolved in deionized water then precipitated with ethanol and centrifuged to collect the precipitate. The polymer was dissolved and re-precipitated under the same conditions then rinsed with ethanol and dried under vacuum. Polymer was then analyzed by ¹H-NMR, ³¹P-NMR, size exclusion high performance liquid chromatography (HPLC), dynamic light scattering (DLS), and Multi-Angle Light Scattering (MALS). The results are as follows.

¹H-NMR (400 MHz, D₂O): δ [ppm]=6.8 (br, 4H, —CH═); 2.6 (br, 2H, Ar—CH₂—); 2.2 (br, 2H, —CH₂—COO); 2.0 (br, 2H, —CH₂—CO—NR₂—); 1.5 (br, 2H, —CH₂—); 1.0 (br, 2H, —CH₂—). Calculated content of carboxylic acid groups—73%.

³¹P-NMR (162 MHz, D₂O): δ [ppm]=−4.0 (br, 2P, —N═P(NH—)₂, —N═P(NH—)(O—Ar)); −18.0 (br, 1P, —N═P(O—Ar)₂). Calculated content of carboxylic acid groups—70%.

DLS: Average hydrodynamic diameter—17 nm.

HPLC: Weight average molecular weight—62 kDa.

MALS: Weight average molecular weight—110 kDa.

### Example 6

This example provides a description of synthesis of poly[(carboxylatoethylphenoxy)(aminoethylpyrrolidinone)phosphazene], 40/60—PCAP-40.

306 mg (1.7 mmol) of MHP was dissolved in 10 mL of diglyme, heated to 120° C. with stirring under nitrogen flow, and kept at this temperature for 30 minutes. The reaction mixture was then allowed to equilibrate at ambient temperature. 77 mg (1.6 mmol) of sodium hydride was suspended in 18 mL of diglyme and added to the reaction mixture dropwise. Stirring was continued for one hour then 14.0 mL (3.2 mmol) of PDCP solution was added dropwise. Temperature was increased to 120° C. and the reaction was allowed to proceed for 2 hours. Heating was stopped and the reaction mixture was allowed to equilibrate at ambient temperature. 0.56 mL (4.0 mmol) of APP in 50 mL of NMP was added dropwise to the reaction mixture while stirring and then kept at ambient temperature. The reaction mixture was then heated to 95° C., 5 mL of 13 M potassium hydroxide was added to the flask, and then heating was turned off. The polymer recovered by decantation, dissolved in deionized water, purified by precipitating in the excess of ethanol three times, and then dried under vacuum. Polymer was then analyzed by ¹H-NMR, ³¹P-NMR, size exclusion HPLC, DLS, and MALS. The results are as follows.

¹H-NMR (400 MHz, D₂O): δ [ppm]=7.0 (br, 4H, —CH═); 3.2-2.8 (br, 6H, —NH—CH₂—, —CH₂—, —NR—CH₂—); 2.7 (br, 2H, Ar—CH₂—); 2.2 (br, 2H, —CH₂—COO); 2.1 (br, 2H, —CH₂—CO—NR₂—); 1.7 (br, 2H, —CH₂—); 1.2 (br, 2H, —CH₂—). Calculated content of carboxylic acid groups—43%.

³¹P-NMR (162 MHz, D₂O): δ [ppm]=0.0 (br, 1P, —N═P(NH—)₂); −3.2 (br, 2P, —N═P(NH—)₂, —N═P(NH—)(O—Ar)), −17.1 (br, 1P, —N═P(O—Ar)₂). Calculated content of carboxylic acid groups—40%.

DLS: Average hydrodynamic diameter—17 nm.

HPLC: Weight average molecular weight—82 kDa.

MALS: Weight average molecular weight—155 kDa.

### Example 7

This example provides a description of synthesis of poly[(carboxylatoethylphenoxy)(aminoethylpyrrolidinone)phosphazene], 20/80—PCAP-20.

The polymer was synthesized as described in Example 6 using the following amounts of the reagents: 171 mg (0.95 mmol) of MHP, 22 mg (0.90 mmol) of sodium hydride, and 0.657 mL (4.8 mmol) of APP. The total volume of diglyme added in the reaction mixture was reduced to 21 mL (not including PDCP solution), and the volume of NMP was increased to 60 mL. Polymer was then analyzed by¹⁻H-NMR, ³¹P-NMR, size exclusion HPLC, DLS, and MALS. The results are as follows.

¹H-NMR (400 MHz, D₂O): δ [ppm]=7.1 (br, 4H, —CH═); 3.4-2.8 (br, 6H, —NH—CH₂—, —CH₂—, —NR—CH₂—); 2.7 (br, 2H, Ar—CH₂—); 2.3 (br, 2H, —CH₂—COO); 1.8 (br, 2H, —CH₂—CO—NR₂—); 1.7-1.2 (br, 4H, —CH₂—, —CH₂—). Calculated content of carboxylic acid groups—22%.

³¹P-NMR (162 MHz, D₂O): δ [ppm]=−2.4 (br, 1P, —N═P(NH—)₂); 1.2 (br, 1H, —N═P(NH—)(O—Ar)). Calculated content of carboxylic acid groups—17%.

DLS: Average hydrodynamic diameter—11 nm.

HPLC: Weight average molecular weight—18 kDa.

MALS: Weight average molecular weight—34 kDa.

### Example 8

This example provides a description of pH dependent membrane active properties of PCAP-20, PCAP-40, and PCAP-70.

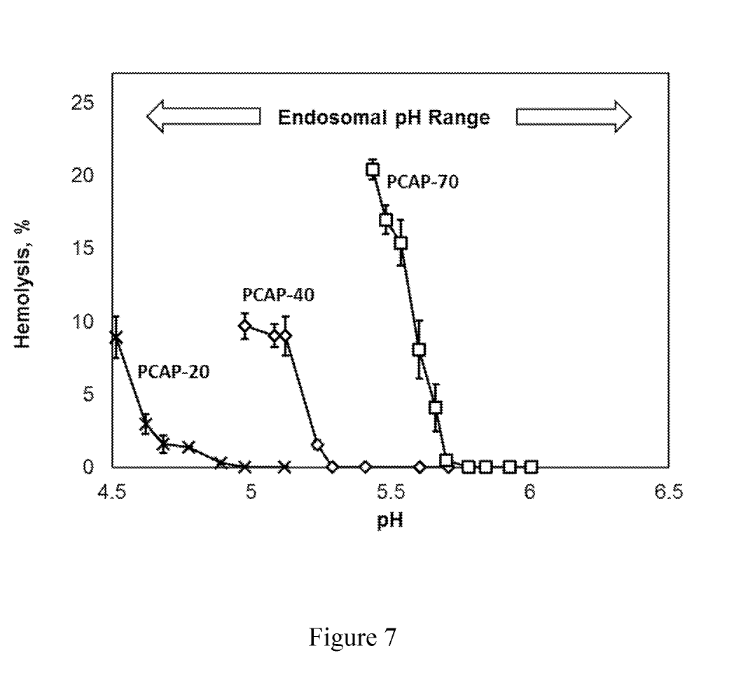

pH dependent membrane disruptive properties of polymers PCAP-20, PCAP-40, and PCAP-70 were investigated as described in the Example 4. The results are shown in FIG. 7.

As seen from the figure, all copolymers show pH dependent membrane activity with a threshold in the range of pH 6.8-4.6, which corresponds to the pH environment of early endosomes.

### Example 9

This example provides a description of hydrolytic degradation of PCAP-20, PCAP-40, and PCAP-70.

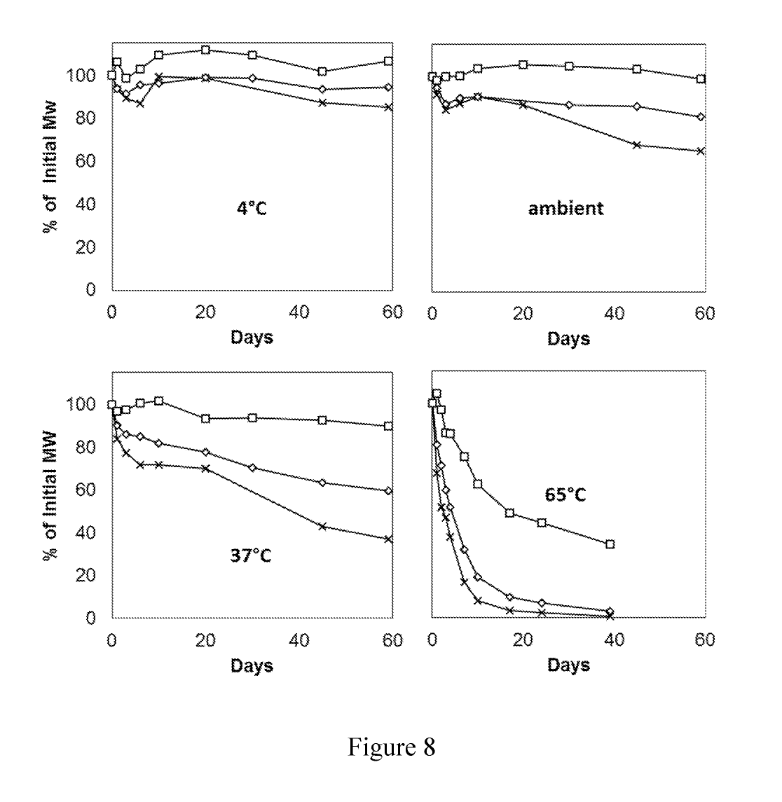

Polymers PCAP-20, PCAP-40, and PCAP-70 were dissolved to a resulting concentration of 0.50 mg/mL in 1× phosphate buffered saline (PBS). Solutions were stored at 4° C., ambient temperature, 37° C., and 65° C. over a period of sixty days. At set time points 0.50 mL sample of each solution was removed for the analysis by size exclusion HPLC. The results are shown in FIG. 8. As seen from the figure, all copolymers demonstrate temperature sensitive hydrolytic degradation. Accelerated degradation conditions (65° C.) demonstrate that polymers PCAP-20 and PCAP-40 show decrease of over 95% of their molecular weight and polymer PCAP-70 over 60% of its molecular weight in a two-month period. Data for 37° C. proves that degradation takes place at a body temperature. Results for 4° C. and ambient temperature, showing either no detectable or minimal degradation, suggest adequate shelf-life of these polymers.

### Example 10

This example provides a description of Protein binding by copolymers PCAP-20, PCAP-40, and PCAP-70.

Polymers were evaluated for their ability to bind a model protein—avidin using asymmetric flow field flow fractionation method (AF4). AF4 is an elution-based method, in which the separation is carried out in a single liquid phase and an external flow of the mobile phase is applied perpendicularly to the direction of sample flow through a channel equipped with semi-permeable membrane. Similar to size-exclusion HPLC, the materials are separated by size, however, as opposed to chromatographic methods, the upper size limit for the analyte can reach as high as 100 μm.

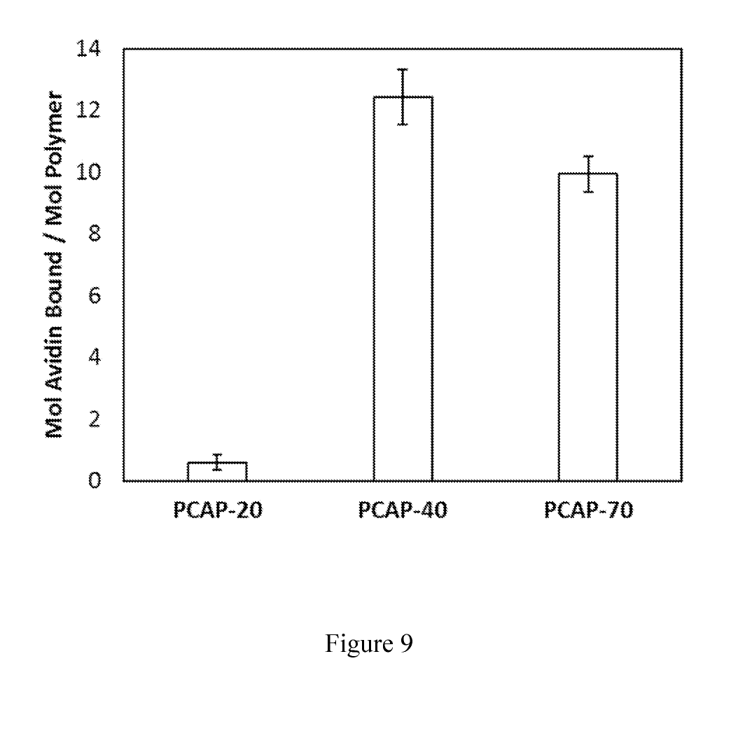

Copolymers, avidin, and their mixtures were dissolved in 1×PBS and analyzed by AF4. Elution profiles were measured at a wavelength of 210 nm. Protein binding was detected by measuring the decrease in avidin peak in the mixture compared to the avidin alone. The results are shown in FIG. 9. As seen from the figure, all copolymers were able to bind avidin, however PCAP-40, and PCAP-70, containing more carboxylic acid groups, displayed highest avidity to the protein. These results demonstrate a potential of the synthesized copolymers as carriers for proteins, including based therapeutics.

### Example 11

This example provides a description of self-assembly of PCAP-20, PCAP-40, and PCAP-70 into nanoparticles.

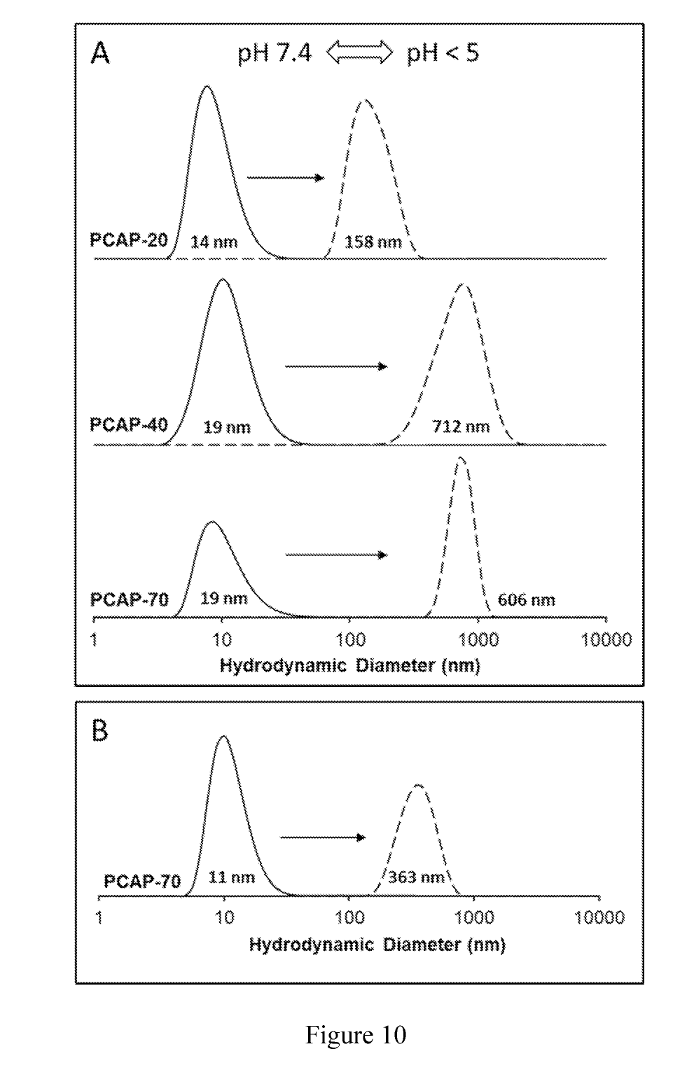

Polymers PCAP-20, PCAP-40, and PCAP-70 were dissolved to a resulting concentration of 0.10 mg/mL in PBS. Dynamic light scattering was performed on resulting polymer solutions. Self-assembly was induced by addition of 0.1 M hydrochloric acid to reduce pH below 5 and dynamic light scattering was performed again. The results are shown in FIG. 10A. As seen in the figure, all polymers form nanoparticles at low pH.

Self-assembly was also induced by addition of spermidine trihydrochloride to a final concentration of 4.5 mg/mL. The results for PCAP-70 are shown in FIG. 10B.

### Example 12

This example provides a description of synthesis of PEGylated PCAP and synthesis of poly[(carboxylatoethylphenoxy)(polyethylene glycol)phosphazene], 85/15—PEG-PCAP-85.

37 μl (0.264 mmol) of triethyl amine was added to 1.2 g (0.24 mmol) of methoxypolyethylene glycol amine, 5 kDa (PEG-NH₂) in 15 mL of diglyme and stirred in a nitrogen filled environment. Low heat was applied to facilitate dissolution. This solution was added to 2 mL (1.6 mmol) of PDCP solution in 13 mL of diglyme while warm and stirring. After 5 hours the solution was allowed to equilibrate at ambient temperature and stirring was continued overnight. 557 mg (3.2 mmol) of MHP in 10 mL of diglyme was heated to 120° C. with stirring under nitrogen flow for 30 minutes then allowed to equilibrate at ambient temperature. This solution was added to 84 mg (3.5 mmol) sodium hydride in 6 mL of diglyme and stirred for 1 hour. Next, the MHP/NaH solution was added to the solution of PEG-NH₂/PDCP and stirred at 120° C. for 3 hours. Heat was reduced to 95° C. and 20 mL of 13M KOH was added, then the solution was allow to equilibrate at ambient temperature and kept in the refrigerator overnight. The polymer was recovered by filtration, dissolved in deionized water, purified by precipitating in acetone twice, and then dried under vacuum. Final purification was achieved using a Superdex preparative column then the polymer was lyophilized. Polymer was then analyzed by ¹H-NMR, ³¹P-NMR, size exclusion HPLC, and DLS. The results are as follows.

¹H-NMR (400 MHz, D₂O): δ [ppm]=6.7 (br, 4H, —CH═); 3.6 (br, 4H, [CH₂—CH₂—O—]ₙ; 2.7-2.4 (br, 4H, Ar—CH₂—, —CH₂—COO). Calculated content of carboxylic acid groups—16%.

³¹P-NMR (162 MHz, D₂O): δ [ppm]=−5.0 (br, 2P, —N═P(NH—)₂, —N═P(NH—)(O—Ar)), −20.1 (br, 1P, —N═P(O—Ar)₂).

DLS: Average hydrodynamic diameter—80 nm.

HPLC: Weight average molecular weight—416 kDa.

1) A multifunctional macromolecular carrier comprising:
i) a hydrophilic macromolecular domain, and
ii) a biodegradable polyphosphazene macromolecular domain comprising one or more ligands having binding affinity to a pharmaceutical agent,
wherein the hydrophilic macromolecular domain and the biodegradable polyphosphazene macromolecular domain are linked through one or more covalent bonds or one or more non-covalent interactions.

2) The pharmaceutical drug carrier of claim 1, wherein the biodegradable polyphosphazene macromolecular domain is a polyphosphazene having the following structure: 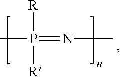
wherein n is an integer from 10 to 500,000,
wherein at least one R or R′ group is a ligand having binding affinity to a pharmaceutical agent.

3) The multifunctional macromolecular carrier of claim 2, wherein the R and R′ groups are at each occurrence in the polyphosphazene selected from ligands having binding affinity to a pharmaceutical agent and ligands having membrane disrupting activity between pH 4.0 and 6.8.

4) The multifunctional macromolecular carrier of claim 2, wherein R and R′ are at each occurrence in the polyphosphazene macromolecular domain are independently selected from: 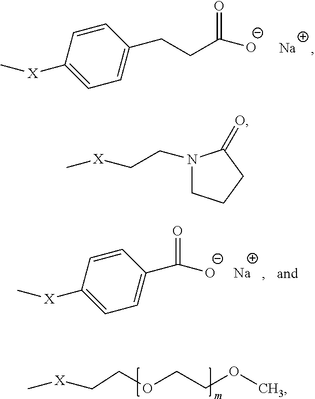
wherein X is −O— or —NH— and m is between 3 and 1,000.

5) The multifunctional macromolecular carrier of claim 1, wherein the hydrophilic macromolecular domain is selected from poly(ethylene glycol), polyvinylpyrrolidone, poly(hydroxypropylmethacrylate), poly(ethylene glycol)-co-poly(propylene glycol), poly(vinyl alcohol), poly[di(methoxyethoxy)phosphazene], poly[di[2-(2-oxo-1-pyrrolidinyl)ethoxy]phosphazene, poly[di(methoxyethoxyethoxy)phosphazene] and combinations thereof.

6) The multifunctional macromolecular carrier of claim 1, wherein the hydrophilic macromolecular domain is poly(ethylene glycol).

7) The multifunctional macromolecular carrier of claim 1, wherein the hydrophilic macromolecular domain is less than or equal to 40 mole percent.

8) The multifunctional macromolecular carrier of claim 7, wherein the hydrophilic macromolecular domain is between 5 and 20 mole percent.

9) The multifunctional macromolecular carrier of claim 1, wherein the multifunctional macromolecular carrier further comprises one or more pharmaceutical agents.

10) The multifunctional macromolecular carrier of claim 9, wherein the pharmaceutical agent is a small molecule drug or combination of small molecule drugs.

11) The multifunctional macromolecular carrier of claim 9, wherein the pharmaceutical agent is selected from nucleic acids, peptide drugs, protein drugs, and combinations thereof.

12) The multifunctional macromolecular carrier of claim 9, wherein the pharmaceutical agent is bound to the multifunctional macromolecular carrier through multivalent non-covalent interactions.

13) A composition comprising one or more multifunctional macromolecular carriers of claim 9.

14) The composition of claim 13, wherein the composition comprises a pharmaceutically acceptable carrier.

15) The composition of claim 13, wherein the composition further comprises one or more excipients that facilitate interactions between the pharmaceutical agent and the multifunctional macromolecular carrier.

16) The composition of claim 15, wherein the excipient comprises spermine, spermidine, or a combination thereof.

17) A method of delivering a pharmaceutical agent to an individual in need of a pharmaceutical agent comprising administering a composition of claim 13 to the individual in need of the pharmaceutical agent.
